(19) United States
(12) Patent Application Publication
(10) Pub. No.: US 2026/0028304 A1
(43) Pub. Date: Jan. 29, 2026

(54) PROCESS FOR CONTINUOUS CATALYTIC HYDROGENATION OF MDA

(71) Applicant: Evonik Operations GmbH, Essen (DE)

(72) Inventors: Hanns Kuhlmann, Sprockhoevel (DE); Armin Matthias Rix, Marl (DE); Niklas Paul, Marl (DE); Lea Wessner, Dortmund (DE); Maria Vargas Gómez, Haltern am See (DE); Tobias Winkler, Duelmen (DE); Florian Boeck, Witten (DE); Daniel Sudhoff, Bergkamen (DE); Christian Lettmann, Coesfeld (DE)

(73) Assignee: Evonik Operations GmbH, Essen (DE)

(21) Appl. No.: 19/276,087

(22) Filed: Jul. 22, 2025

(30) Foreign Application Priority Data
Jul. 26, 2024 (EP) 24191095.9

(51) Int. Cl. C07C 209/72; C07C 209/86

(52) U.S. Cl. CPC C07C 209/72; C07C 209/86; C07C 2601/14

(57) ABSTRACT

A plant for hydrogenation of methylenedianiline with a hydrogen donor has a conditioning unit for the reactants, a reactor unit for synthesis of PACM and a separation unit. The conditioning unit has at least part of the length of the (feed) conduits for reactants1, reactant2 and at least one solvent, at least one heat exchanger in at least one (feed) conduit, at least one mixer for mixing the reactants and/or at least one reactant with at least one solvent. The reactor unit has at least one fixed bed reactor as main reactor with an immobile catalyst packing. The separation unit has at least a first separation stage for removal of the at least one solvent and a second separation stage for separation of the reactant and by-products from the PACM product. The hydrogenation of MDA is conducted in a corresponding process.

### CROSS-REFERENCE TO RELATED APPLICATIONS

This patent application claims priority to European Patent Application No. 24191095.9, filed on Jul. 26, 2024, in the European Patent Office, the entire disclosure of which is hereby incorporated by reference herein.

### BACKGROUND OF THE INVENTION

### Field of the Invention

The invention relates to a plant and to a process for continuous catalytic hydrogenation of MDA, especially for production of methylenebis(cyclohexylamine), such as 4,4′-diaminodicyclohexylmethane (PACM) in particular.

### Description of Related Art

Processes for hydrogenation of organic compounds, especially for hydrogenation of aromatic compounds to the corresponding cyclohexane derivatives, are already known from the prior art.

Methylenebis(cyclohexylamine) is a cycloaliphatic amine that is solid or liquid under standard conditions (SATP) and is typically produced by liquid phase hydrogenation of MDA. The acronym MDA was introduced historically as an abbreviation for the product mixture which is formed in the reaction of aniline and formaldehyde and comprises mainly “methylenedianiline” (diaminodiphenylmethane), and is still used to describe the process product, which is now produced on an industrial scale. The hydrogenation product, comprising mainly methylenebis(cyclohexylamine), is therefore often also referred to as H12MDA.

Because of the process for production thereof, MDA is typically a mixture of different diaminodiphenylmethanes. It consists mainly of 4,4′-diaminodiphenylmethane. However, 2,4′ and 2,2′ isomers may also be present. MDA may also contain reaction products having three or more aromatic rings that are formed in the reaction of aniline and formaldehyde, in particular those having three or more phenyl rings. These reaction products having three or more aromatic rings are also referred to as polynuclear compounds.

Because of the high proportion of 4,4′-diaminodiphenylmethane in the MDA used, commercially available methylenebis(cyclohexylamine) is mostly 4,4′-diaminodicyclohexylmethane or bis(para-aminocyclohexyl) methane. Because of the potential presence of the corresponding 2,4′- and 2,2′-diaminophenylmethane isomers in the MDA, 2,4′-diaminodicyclohexylmethane and 2,2′-diaminodicyclohexylmethane may also be present in methylenebis(cyclohexylamine). In addition, hydrogenated MDA may also contain (optionally partly) hydrogenated polynuclear compounds as well as methylenebis(cyclohexylamine).

U.S. Pat. No. 5,578,546 A discloses that a process for producing methylenebis(cyclohexylamine) was first described in 1947 and converted to industrial scale in 1965. Hydrogenation of MDA is highly exothermic. For example, WO 2010/069484 A1 indicates an enthalpy of reaction of −1600 kJ/mol.

As a result of hydrogenation, various diastereoisomers are formed depending on the process. The 4,4′-diaminodicyclohexylmethane (PACM) product derived from 4,4′-diaminodiphenylmethane may take the form of trans/trans, cis/cis and cis/trans isomers and is therefore generally a mixture of these isomers with different proportions. The melting point of the compound rises with rising trans/trans content. Therefore, the fields of application vary significantly depending on the isomer content: While methylenebis(cyclohexylamine) qualities with a low trans/trans content (e.g. 10-30% by weight) are used in the field of amine and isocyanate crosslinkers, especially in the field of two-component resins, qualities with a high trans/trans content (e.g. ≥48% by weight) are mainly used as regulator in polyamide compounds. The production of products with a low trans/trans content in particular is a challenge, since the thermodynamic equilibrium, as described in U.S. Pat. No. 3,636,108 A, is in the range of significantly higher trans/trans contents (up to 51.2%). In addition, U.S. Pat. No. 2,606,925 A shows that the equilibrium can be shifted retrospectively by prolonged heat treatment in the direction of a higher proportion of trans/trans isomer.

The composition of the hydrogenation product also depends on the composition of the MDA used: MDA is often used in qualities from MDA50 to MDA100, with the number between 50 and 100 indicating the content of diaminodiphenylmethanes in the MDA mixture. MDA50 is an MDA quality which, as explained above, for process-related reasons, contains about 50% by weight of diaminodiphenylmethanes and 50% by weight of polynuclear compounds. The individual polynuclear compounds can be referred to as trinuclear compounds, tetranuclear compounds, etc. according to the number of aromatic nuclei present. MDA50 is the quality produced in the highest volume and is mainly processed to methylene dicyclohexyl diisocyanate (MDI). MDA100 is pure MDA or diaminodiphenylmethane without polynuclear compounds. MDA85 or MDA90 are other grades of medium purity available on the market. When patent specifications relating to the process for production of methylenebis(cyclohexylamine) address the purity of the MDA quality, the quality in question is usually MDA100 (e.g. CN 110204447 B). US 2005/261525 A1, by contrast, prefers the hydrogenation of MDA50. The hydrogenated oligomeric amines obtained here as high boilers are suitable as crosslinkers having particularly low vapour pressures for a range of specialty applications, as shown by US 2004/162409 A1.

The content of (possibly partly) hydrogenated polynuclear compounds in the product decreases in the order of the reactants MDA50, MDA85, MDA90, MDA100, since the content of polynuclear compounds decreases from MDA50 to MDA100.

WO 2009/153123 A1 discloses a continuous process and a reactor for hydrogenation of organic compounds in a polyphasic multistage system in the presence of a homogeneous or heterogeneous catalyst. Catalysts proposed include precious metals such as platinum, palladium, ruthenium and rhodium, or other transition metals such as molybdenum, tungsten and chromium. It is possible here for the heterogeneous catalysts to be disposed on support materials, for example carbon, aluminium oxide, silicon dioxide, zirconium dioxide, zeolites, aluminosilicates or mixtures of these support materials. Substrates used in this process are preferably aromatic compounds containing amino substituents, for example MDA, polymer MDA, aniline, 2,4-diaminotoluene, 2,6-diaminotoluene, o-phenylenediamine, etc. The heterogeneous catalysts are used in suspension.

DE 19533718 A1 discloses a process for hydrogenation of aromatic compounds in which at least one amino group is bonded to an aromatic nucleus. For this purpose, it is possible to use a heterogeneous catalyst containing ruthenium and optionally at least one metal of transition group I, VII or VIII. The support material used is, for example, aluminium oxide, silicon dioxide, titanium dioxide or zirconium dioxide, preferably aluminium dioxide or zirconium dioxide. Only a catalyst containing ruthenium on the support material aluminium oxide is given as an example, and not zirconium oxide.

EP 1337331 A1 discloses a process for catalytic hydrogenation of aromatic or heteroaromatic amines, wherein ruthenium acts as the active metal and the catalyst contains at least one additional metal of transition group I, VII, or VIII, and these are applied to a support material.

Aromatic compounds used here include 4,4′-MDA and isomers thereof. EP 0111238 A1 also discloses a process for catalytic hydrogenation of 4,4′-MDA, characterized in that the hydrogenation is effected in the presence of supported ruthenium in the presence of nitrates and sulfates of the alkali metals and nitrates of the alkaline earth metals. A comparable process is disclosed in EP 1366812 A1, where support materials mentioned include aluminium oxide, silicon oxide, titanium oxide and zirconium oxide.

Further processes for hydrogenation of organic compounds are disclosed by WO 2011/003899 A1 and WO 2009/090179 A1. What are disclosed here are processes for hydrogenation of aromatic amines with hydrogen in the presence of an Ru catalyst containing zirconium oxide support material inter alia.

DE 101 19 135 A1 discloses a plant and a process for production of diaminodicyclohexylmethane (PACM) with a proportion of trans, trans-4,4′-diaminodicyclohexylmethane of 17% to 24%, where this is achieved by hydrogenation of diaminodiphenylmethane (MDA) in the presence of a pulverulent catalyst. The hydrogenation is conducted here in a continuously operated suspension reactor, in particular in a cascade of multiple series-connected suspension reactors, such that an MDA conversion of at least 95% is achieved, based on the amount of MDA used.

EP 1 519 912 B1 discloses a plant and a process for production of 4,4′-diaminodicyclohexylmethane (4,4′-HMDA), wherein the reaction is effected by means of heterogeneous, catalytic hydrogenation of a substance mixture comprising 4,4′-diaminodiphenylmethane (4,4′-MDA) as the main component and its mono-N-methyl derivative as a secondary component with elevated selectivity with regard to the hydrogenation of 4,4′-MDA at a temperature in the range from 50 to 220° C. and a hydrogen pressure in the range from 1 to 30 MPa. For this purpose, EP 1 519 912 B1 suggests ending the hydrogenation prior to attainment of a conversion of 4,4′-MDA to 4,4′-HMDA of 99%, especially in the range from 90% to 98.9%.

Finally, EP 2 883 863 B1 (D1b) discloses a process and a plant for hydrogenation of 4,4′-methylenedianiline (MDA) and/or polymer MDA with hydrogen in the presence of a catalyst. A catalyst proposed here is ruthenium applied to a zirconium oxide support material. With regard to the reactor, EP 2 883 863 B1 refers to the reactor or the reactor concept of WO 2008/015135 A1 (D1a). Said WO 2008/015135 A1 discloses a continuous process and a plant for hydrogenation of diisononyl phthalate to diisononyl cyclohexane-1,2-dicarboxylate (DINCH), wherein DINP is hydrogenated as a mixture in an organic solvent with hydrogen at a pressure of up to 325 bar. What is proposed here is series connection of two fixed bed reactors each with an immobile fixed bed. In order to dissipate the heat of reaction from the two reactors, it is proposed that a substream of the stream of matter be recirculated and thereby cooled downstream of the second fixed bed reactor. A comparable reactor concept of series-connected fixed bed reactors for hydrogenation of is also known from EP 1 566 372 B1.

This concept is disadvantageous in that the circulation of a product substream can lead to increased formation of unwanted by-products and reduces plant performance, causing increased energy costs for the recycling and cooling of the recycling stream.

As explained, the need for PACM, for example, with different proportions of the respective isomers is dependent on the intended use or the subsequent products. For example, PACM qualities with a low trans/trans content of 10% to 30% by weight are preferred in the field of amine and isocyanate crosslinkers, especially in the field of formulation of 2-component resins, and PACM qualities with a high trans/trans content exceeding 48% by weight are mainly used as regulator in polyamide compounds. The percentages by weight mentioned are based here on the PACM isomer mixture as such. The production of products with a low trans/trans content in particular is a process engineering challenge, since the thermodynamic equilibrium, as described in U.S. Pat. No. 3,636,108 A, is in the range of significantly higher trans/trans contents of up to 51.2% by weight. Furthermore, it is known from U.S. Pat. No. 2,606,925 A that the equilibrium of the PACM isomers is subsequently shifted in the direction of a higher proportion of trans/trans isomer by prolonged heat treatment.

### SUMMARY OF THE INVENTION

It is thus an object of the present invention to provide an improved plant and an improved process which is improved in terms of product conversion and energy efficiency and in particular enables the production of defined proportions of the respective isomers in the isomer mixture.

### BRIEF DESCRIPTION OF THE DRAWINGS

FIG. 1 shows a plant as a process flow diagram.

FIG. 2 shows a first embodiment of a two-stage expansion.

FIG. 3 shows a further embodiment of a two-stage expansion.

FIG. 4 shows a variant of the embodiment according to FIG. 3.

FIG. 5 shows a further embodiment of the reaction unit.

FIG. 6 shows a further embodiment of the reaction unit.

### DETAILED DESCRIPTION OF THE INVENTION

The object is achieved in accordance with the invention by a plant according to the features of embodiment 1 and a process according to the features of embodiment 13.

What is provided here is a plant for continuous catalytic hydrogenation of methylenedianiline (MDA; reactant1) with a hydrogen donor (reactant2), especially a gaseous hydrogen donor, preferably hydrogen (H2),
comprising a conditioning unit for the reactants, a reactor unit and a separation unit, wherein
the conditioning unit comprises at least (feed) conduits for reactants1, reactant2 and at least one solvent, at least one heat exchanger in at least one (feed) conduit, at least one mixer unit for mixing the reactants and/or at least one reactant with at least one solvent;
the reactor unit comprises at least one fixed bed reactor as main reactor with an immobile catalyst package, wherein the at least one main reactor comprises
   a first flow pathway for the mixture of matter through the immobile catalyst packing and
   another separate closed flow pathway, or media circulation system, for a heat exchange medium outside the catalyst packing, i.e. the first flow pathway, and
   wherein the media circuit incorporates a heat exchanger;
the separation unit comprises at least
a first separation stage for (essentially) removal of the solvent and
a second separation stage for (essentially) separation of the at least one reactant and/or at least one by-product from the product, wherein the separation unit comprises in the first separation stage at least one pressure control unit and a separation tank having a condensation unit for the solvent, wherein a (return) conduit for the solvent leads from the at least one condensation unit of the first separation stage to the conditioning unit.

The plant is preferably used for continuous catalytic production of methylenebis(cyclohexylamine) as product, especially for production of 4,4′-diaminodicyclohexylmethane (PACM), of the formula (I)

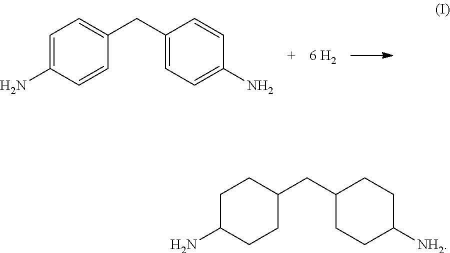 [SMILES: Nc1ccc(Cc2ccc(N)cc2)cc1 ; NC1CCC(CC2CCC(N)CC2)CC1]

The liquid stream of matter coming from the separation tank is directed via a (feed) conduit to the first separation column in the first separation stage of the separation unit. Advantageously, in one embodiment of the plant, it may be the case that a pressure control unit is provided in the (feed) conduit from the separation tank to the first column. In this way, the reactor unit may be operated at a first, high pressure level, and the first separation column of the first separation stage at a second, lower pressure level, where the separation tank may be operated at an intermediate level.

Furthermore, it may be advantageous when a (forward) heat exchanger is provided upstream of the first separation column of the first separation stage, especially also downstream of the pressure control unit or between the pressure control unit and the first separation column.

In a particularly advantageous embodiment, an energy coupling is provided in order to operate the condenser downstream of the separation tank, a condenser of a separation column of the second separation stage or the (circulating) heat exchanger in the media circuit of the at least one main reactor interconnected in heat exchange with the (forward) heat exchanger of the first separation column. The energy coupling can be effected by conduction of media and a series interconnection of the respective heat exchangers or by integrated energy coupling in a (structurally) single heat exchanger. Integrated energy coupling has the advantage, if spatially implementable within the plant, that only a temperature gradient for heat transfer has to be overcome. It is thus possible by energy transfer to achieve a parallel rise in the (feed) stream of matter upstream of the first separation column of the first separation stage, which is at a temperature level of about 85 to 95° C. downstream of the upstream pressure control unit in the (feed) conduit. Since the main reactor, in the course of operation, is operated at a constantly rising temperature in order to compensate for the falling catalyst activity, a constantly rising amount of energy can be released to the (feed) stream of matter upstream of the first separation column in parallel. As a result, there is likewise a steady drop in the energy demand in the bottoms circuit, or the heat exchanger of the first separation column incorporated therein.

With the separate postreactor, especially the adiabatic postreactor, an optimized control of the process and the plant has been enabled, whereby selectivity becomes possible because of the variance of the inlet temperature of the postreactor from the main reactor (outlet). Typically, the inlet temperature of the postreactor may be provided at the same or a slightly lower temperature, which may be up to 30° C. below the outlet temperature of the main reactor. Thus, at least temporarily, an increase in temperature in the (adiabatic) postreactor of 20° C. to 40° C., typically of 20° C. to 30° C., may be allowed, and hence the desired product quality (isomer ratio) can be specifically adjusted. In this way, a very low and a very exact proportion of trans/trans isomers in the isomer mixture can be established. The main reactor can be operated at a very low temperature level such that the trans/trans content of the PACM in the stream of matter (outlet of the main reactor) is about 13% to 20% by weight, with about 13% being achievable in the case of a new or regenerated catalyst and 20% by weight in the case of a catalyst after prolonged use (shortly before replacement/regeneration). Depending on the main reactor phase, it may be useful at least temporarily if the inlet temperature of the postreactor is 5 to 20° C. higher than that of the main reactor.

The main reactor that has been freshly filled with catalyst or regenerated, with the strongly exothermic reaction therein, is operated largely isothermally, although this should not be understood in the ideal sense. Even though the main reactor is referred to in the present context as being “isothermal”, this ideal state is only achieved to a limited degree in industrial use, and so a temperature gradient of about 5 to 10° C. in radial direction and also in flow direction develops within the main reactor owing to incomplete heat dissipation.

The postreactor is charged via the upstream heat exchanger with a somewhat higher temperature so as to result in the desired residual reaction and isomerization to give the final desired trans/trans ratio of 17% to 23% by weight, for example. The postreactor with the weakly exothermic residual reaction therein is operated in a largely adiabatic manner, although this should not be understood in the ideal sense.

As time advances, the catalyst activity decreases until replacement or regeneration is required. For this purpose, in parallel, the operating temperature is raised by the controlling of the cooling circuit, i.e. in particular by cooling to a lesser degree via the heat exchanger incorporated in the cooling circuit in order to keep conversion and selectivity at a largely constant level overall. As a result, the isomer ratio is shifted, toward higher trans/trans contents in the product. It has been found here to be very advantageous that the temperature in the feed of the postreactor can be controlled autonomously by means of the upstream heat exchanger, in particular in the same sense. Thus, as the operating temperature of the main reactor rises over the service life of the catalyst, the feed temperature in the stream of matter of the postreactor is lowered.

The liquid stream of matter coming from a flash separation tank, referred to hereinafter as separation tank (also called “flash vessel”), is directed via a conduit to the first separation column within the first separation stage of the separation unit. It is a feature of the separation tank that the incoming stream of matter is separated into a vapour phase (solvent, solvent-rich) and a liquid phase (solvent-depleted) by expansion (lowering the pressure) and that both phases are present in the separation tank in regular operation. The separation tank may additionally have a bottoms circulation system with integrated heat exchanger or be connected thereto in order to increase the removable vapour content beyond the pressure-related fraction by heating the liquid phase. Furthermore, a separation tank may comprise internals or random packings, in order in particular to prevent entrainment of droplets that have only been incompletely depleted of solvent, if at all.

Advantageously, in one embodiment of the plant, it may be the case that a pressure control unit is provided in the conduit from the separation tank to the first column. This allows the reactor unit to be operated at a first, high pressure level, and the first separation stage of the separation unit at a second, lower pressure level. What is thus meant in the present context by separation tank (flash vessel) is a device whereby a phase separation is caused essentially by expansion. What is meant here by separation column, by contrast, is an apparatus in which a separation into a vapour phase and liquid phase takes place essentially by supply of energy, in particular by incorporating a tops circulation system in which at least a portion of the liquid condensed out is directed back into the column at the top.

What is meant in the present context by “an increase in efficiency”, unless stated otherwise, is a lower energy consumption. The reference point will be apparent from the context and may be based on the plant, the plant section respectively described or the improved apparatus, for example via integration of two heat exchangers into a single one.

Advantageously, the mixing unit is formed in two parts and comprises, for example, a mixing apparatus and a gas saturator. The mixing apparatus may especially be a dynamic or static mixer suitable for intimate association of MDA (reactant1) with the solvent. The gas saturator may especially be a small column or a tank in which there are suitable internals for intensive dissolution of the H2 gas supplied under a high pressure in the MDA/solvent stream of matter and/or for homogeneous distribution thereof prior to entry into the main reactor. The H2 gas pressure is advantageously 70 bar to 100 bar.

The term “immobile catalyst packing” or “immobile catalyst” refers to any form of a local catalyst that does not flow or move with the stream of matter, such as, in particular, catalyst beds (pellets or coated carrier bodies) or fixed internals coated with catalyst material. Advantageous installation parts having a catalyst coating may be, for example, grids, plates or other bodies arranged in the main reactor.

In the present context, an “XY unit” and/or “XY stage” always also means at least one corresponding apparatus, device or the like which is included in the respective unit or stage. This means, for example, a mixing unit/stage comprising this at least one mixer/mixing device.

What is meant in the present context by “heat exchange” or “heat exchanger” is always indirect heat exchange and corresponding designs with closed material and media conduits, in the absence of any explicit description to the contrary.

The “condensation unit” of the first separation stage refers to a single heat exchanger or a group of heat exchangers which are used for at least partial condensation and/or cooling of the fractions of light boilers derived via the top conduit(s). A “condensation unit” referred to as such need not be a (closed) structural unit. Thus, to some degree, the term “condensation unit” and a single “heat exchanger” are also used synonymously.

The stage referred to as “first separation stage” is determined in particular in that, and has corresponding apparatuses and conduits such that, the solvent is (specifically) separated from the product-rich stream of matter and is advantageously also returned to use in the reactor unit and/or the conditioning unit. In an analogous manner, the stage of the separation unit referred to as the “second separation stage” means that it is thus determined, and has corresponding apparatuses and conduits, in order to separate the product from by-products and reactants, in particular MDA, and purify the product. In this case, the first and second stages are naturally not strictly separated, and there may be an overlap region or an apparatus in the transition region in which both solvent and at least one by-product or at least one reactant is separated from the product, especially PACM.

In the present context, what is meant by the “removal of solvent” in the first separation stage and “separation of at least one reactant and/or at least one by-product from the PACM product” is that the removal/separation does not mean an absolute delimitation of the separation stages, but in each case affects “essentially” only the substance(s) mentioned.

What is meant in the present context by the term “reactant mixture” is the mixture of matter present on entry into the (first) main reactor, i.e. the mixture of all reactants, solvents, auxiliaries etc. In and downstream of the at least one reactor, the flowing mixture of matter in any degree of reaction or subsequent purification is referred to as “stream of matter” or “mixture of matter”, in some cases with addition of adjectival descriptions such as “product-rich” or “solvent-rich”. However, the physical composition of the stream of matter at each site in the plant is also obvious to the skilled person by virtue of that site in the plant and upstream plant components, and in particular the process engineering apparatuses of the plants. The pure substances, for example the PACM product, and the LB and HB by-products, are named and identified separately. “LB” here stands for “low boilers”, a valuable mixture of matter that is separated separately from the stream of matter and from the product and has a low boiling point of about 240° C. to 290° C. Analogously, “HB” stands for “high boilers”, a valuable mixture of matter that is separated separately from the stream of matter and from the product and has a high boiling point of >350° C.

What are described in particular in the present context are a plant and a process for production of methylenebis(cyclohexylamine) as product, especially for production of 4,4′-diaminodicyclohexylmethane (PACM), by catalytic hydrogenation of methylenedianiline. The primary product here is PACM, which is hydrogenated from 4,4′-diaminodiphenylmethane (4,4′-MDA; primary fraction of reactant1), especially with the low trans/trans isomer ratio mentioned, and so the plant and the process serve, and are suitable, in particular for production of 4,4′-diaminodicyclohexylmethane (PACM) by continuous catalytic hydrogenation of 4,4′-diaminodiphenylmethane (4,4′-MDA). The additional fractions of 2,4′-MDA and 2,2′-MDA of the MDA present are converted at least in small proportions in parallel to reaction products, where these generally remain in the product mixture. In addition, other by-products can advantageously likewise be purified and separated off by this process or this plant in the second separation stage of the separation unit, especially in at least one separation column. These are regularly secondary (valuable) products, herein described as and meaning essentially high boilers (HB) and low boilers (LB).

The solvent is advantageously from the following group of substances: cyclohexane, dioxane, tetrahydrofuran (THF), cyclohexylamine, dicyclohexylamine, methanol, ethanol, isopropanol, n-butanol, 2-butanol, 2-methoxy-2-methylpropane (MTBE) or methylcyclohexane or a mixture thereof. Advantageously, the solvent, especially THF, is fed in excess into the MDA, such that the ratio of the mass flow rates of solvent, especially THF, to MDA at the inlet of the main reactor is advantageously in the range from 1.0 to 8.0, especially in the range from 2.0 to 6.0.In one embodiment of the plant, it may be advantageous that the reactor unit comprises a first main reactor and at least one downstream permanent series-connected postreactor. The particular advantage of the postreactor and its inlet-side temperature control of the stream of matter is that this enables optimized control of the selectivity of the proportions of isomers, because of the preferably higher inlet temperature that differs from the first main reactor.

Advantageously, the postreactor is also a fixed bed reactor, or reactor with an immobile catalyst, for example a catalyst bed or catalyst-coated internals.

Advantageously, the immobile catalyst comprises ruthenium, has been doped with ruthenium or is formed therefrom. In an advantageous process variant, especially for achieving a low proportion of trans/trans isomers in the isomer mixture, the main reactor is at a temperature of 90 to 140° C., ideally 95 to 135° C.

It has been found to be particularly advantageous when the ratio of the catalyst masses of the main reactor to the postreactor is in the range from 1.2 to 2, preferably 1.3 to 1.4 and ideally 1.35. It has been found that, surprisingly, it is sufficient to regulate the highly exothermic reaction at the start of the reaction in the main reactor by means of an intensive cooling circuit, and only to adjust the feed temperature in the inlet to the postreactor such that the moderate temperature rise of about 30 to 35° C. in the postreactor from the inlet to the outlet has only a limited and readily reproducible influence on the trans/trans isomer content in the stream of matter or in the product, as already set out.

In a further embodiment of the plant, it may be advantageous that the reactor unit comprises a further main reactor in the form of a fixed bed reactor comprising
a first flow pathway for the mixture of matter and
a further (closed) flow pathway for a heat exchange medium, and where a valve unit is provided upstream of the two main reactors in the (feed) conduit, by means of which the volume flow rate of the reactant mixture is divisible, conductable and/or completely switchable between the first main reactor and the further main reactor, and wherein the two main reactors are connected
each to one heat exchanger or
collectively to one heat exchanger
   for the further (closed) flow pathway.

In this case, the two main reactors connected in series are identical or essentially identical in design. In particular, the two main reactors have such dimensions and/or corresponding internals such that the same or substantially the same mass and/or volume of catalyst is present or accommodatable.

The advantage of these two main reactors is that further thermal decoupling of the first reaction phase with very strong exothermicity, the second reaction phase with medium exothermicity and the postreaction with very low exothermicity is possible. Further advantages are that, in the case of the same plant performance, the individual main reactor has smaller dimensions and can therefore be thermally controlled more easily and more homogeneously. At the same time, plant performance is increased because, in the case of maintenance, for example a catalyst changeover, the plant does not have to be shut down completely. For this purpose, the two main reactors are interconnected in such a way that the mixture of matter can also flow through each alone, bypassing the respectively other main reactor (bypass 1).

The hydrogenation in the (isothermal) main reactor with significant cooling allows significant limitation of temperature-induced isomerization. In the (adiabatic) postreactor, a sufficient temperature level is then established in a controlled manner in the incoming stream of matter, especially via heat exchange, and hence isomerization is permitted to specifically afford an on-spec trans/trans content in the product. In a purely isothermal mode of operation of a single main reactor without a postreactor, what would at first be obtained would be excessively low trans/trans contents and a high proportion of unconverted MDA. By virtue of the option of allowing controlled adiabatic hydrogenation of the stream of matter, it is possible to advantageously control the evolution of temperature and hence isomerization in the postreactor.

In a further-improved variant, the interconnection is such that the postreactor can be bypassed in the case of operation of at least one main reactor (bypass 2), but can especially be bypassed in the case of operation of the two main reactors connected in series. In the bypass 2 interconnection variant, the main reactor through which the flow passes second in flow direction at least temporarily assumes the function of the postreactor, such that the plant can be operated without or largely without a drop in performance and/or changes in product quality, especially in the respective proportion of isomers in the isomer mixture.

In a further embodiment of the plant, it may be advantageous that at least one common heat exchanger is disposed in the conduit between the at least one main reactor and the second main reactor. This common heat exchanger is disposed in a central branch of both coolant circuits. The guiding and interconnection of the conduits here is such that, downstream of the common heat exchanger, the cooling medium is first introduced into the upstream (first) main reactor in which the more strongly exothermic reaction proceeds. The already heated cooling medium is then fed via a conduit into the downstream second main reactor, such that the latter is charged with a feed temperature different from the first main reactor.

The advantage is that it was observed that, surprisingly, in particular, very exact control of the first, highly exothermic reaction phase is crucial for product quality, and so it is possible to dispense with the construction work and control complexity involved in a further, completely independent second cooling circuit. Another reason for this is in particular because complete reaction can be ensured and controlled via control of the feed temperature of the postreactor connected in series downstream of the two main reactors; in particular, the desired low trans/trans isomer content can be established.

In a plant variant with even better controllability, it may be the case that a heat exchanger (postcooler) which is switchable and controllable as required is disposed in the respective (cross-) conduit of the cooling circuits by which the coolant outlet of the first reactor is connected to the coolant inlet of the second reactor.

In the present context, essentially the following energy couplings (EC) are considered, such as integrated energy coupling or direct energy coupling, where integrated EC means integrated energy coupling, subdivided into
a. integrated material-based energy coupling (integrated material-based EC) of at least two streams of matter in a heat exchanger in indirect heat exchange, i.e. structural integration in a single heat exchanger (apparatus), meaning that, where two heat exchangers were formerly provided, both heat transfer functions of a structural unit (heat exchanger) are integrated,
b. integrated media-based energy coupling (integrated media-based EC) of at least two heat exchange media in a heat exchanger in indirect heat exchange, meaning structural integration in a single heat exchanger (apparatus).

The aforementioned ECs may be designed as direct energy coupling (direct EC) in that a serial EC or serial interconnection of at least two heat exchangers is provided.

In a further embodiment of the plant, it may be advantageous that the separation unit comprises a separation tank which is connected to the main reactor or the postreactor via a conduit such that the mixture of matter can be introduced into the latter. In particular, the last reactor in flow direction is connected to the separation tank. The separation tank here comprises:
a tops outlet,
a base/bottoms outlet and
a heatable bottoms circulation system having and/or connected to at least one heat exchanger, where the (tops) conduit incorporates a condensation unit for at least partial liquefaction (condensation) of the solvent, and where the following are disposed downstream of the condensation unit:
a (collection) vessel and/or
a connecting unit/node to or into the (return) conduit for the solvent. The bottoms circulation system also includes a pump.

The bottoms circulation system of the separation tank is operated at a temperature of 130° C. to 150° C., ideally 135 to 145° C. The great advantage of the separation tank, which is very simple in terms of construction and control, is that the boiling temperature of the mixture is likewise lowered by the lowering of pressure, such that heating in the separation tank is only necessary up to that lowered boiling temperature. Furthermore, by virtue of this measure, about 90% of the solvent present in the reactant mixture, in particular THF, is already removable. There is no need for this purpose for a tops circuit or return stream, as in a column. The stream of matter passed onward to the first separation column thus advantageously has only a remaining solvent concentration of about 20% to 40% by weight, ideally 25% to 35% by weight. It is thus possible for the first separation column to be of smaller design, and operable in a more energy-saving manner owing to the lower mass of the stream of matter.

This expansion step in a two-or multistage interconnection of separation tanks with direct heat integration of the first separation stage can reduce the energy requirement of this expansion step by 40% or more.

In one variant of this embodiment of the plant and of the process described below, it may be advantageous when the separation unit comprises at least one further (second) separation tank disposed downstream of the first separation tank, where the bottoms outlet of the first tank is connected to the inlet (feed) of the second separation tank, where the second separation tank also comprises a tops outlet, a base/bottoms outlet and a heatable bottoms circulation system having at least one heat exchanger. In this case, a (tops) conduit leads from the (tops) outlet of the second tank to the condensation unit and/or into the (tops) conduit of the first tank. Advantageously, a pressure control unit is provided in the conduit leading to the inlet of the second tank, such that the first tank is operable at a first temperature and a first pressure, and the second tank is operable at a second temperature lower than the first temperature and a lower pressure compared to the first pressure. The pressure control unit is advantageously a valve or throttle controllable by open-loop and closed-loop control. For formation and delimitation of the pressure levels in the two separation tanks, an analogous pressure control unit may advantageously be provided in the tops conduit.

In a further embodiment of the plant, it may be advantageous that the condensation unit of the first separation stage comprises at least two heat exchangers, where the connecting unit/node for the (tops) conduit of the second separation tank to the (tops) conduit of the first separation tank is disposed between the two heat exchangers of the condensation unit.

In this case, a pressure control unit is advantageously provided in the tops conduit from the first separation tank to the collection vessel upstream of the conduit node at which the tops conduit of the second separation tank opens into the tops conduit leading to the collection vessel. By means of these two heat exchangers of the condensation unit in the conduit leading to the collection vessel, it is possible to keep the respective stream of matter at the respective temperature level and pressure level of the associated separation tank.

In a further embodiment of the plant, subsequent interconnection of the heat exchangers and thus onward conduction of the medium may be advantageous. An advantageous interconnection may exist when a (media) conduit from the (media) outlet of the (first) condensation unit in the tops conduit of the first separation tank, especially of at least one of the heat exchangers, leads to the inlet of the (bottoms) heat exchanger of the second separation tank. In this way, in particular, the complete energy requirement of the heat exchanger incorporated in the bottoms circuit of the second separation tank can be met.

In an advantageous process regime, the temperature level in the (tops) conduit of the first separation tank is 110 to 130° C., ideally 115 to 120° C., at a pressure of 3 to 5 bar, ideally of 3.5 to 4.5 bar. After condensation in the evaporator unit of the second separation tank, the temperature of the stream of matter in the (tops) conduit is lowered by about 8 to 20° C.

The temperature in the (tops) conduit of the second separation tank is 80 to 100° C., ideally 85 to 95° C., where the pressure is in the range from 1.0 to 2 bar, ideally 1.1 to 1.5 bar.

In an alternative embodiment of the plant with respect to the two-stage separation tanks, it may be advantageous when the separation unit, at the outset in the first separation stage, comprises and/or is essentially formed from at least two evaporators as separation tank, which are connected in series, where at least one heat exchanger designed as a condenser is provided as condensation unit in the (tops) conduit of at least one of the two evaporators. In an advantageous embodiment, at least one of the evaporators may take the form of what is called a kettle-type evaporator; in particular, both evaporators advantageously take the form of kettle-type evaporators. In addition, the following are disposed in the (tops) conduit downstream of the heat exchanger:
a (collection) vessel and/or
a connecting unit/node to/into the (return) conduit for the solvent.

These evaporators comprise two portions. A heat exchanger portion (WT portion) and a tank portion (K portion), where the WT portion protrudes into or is disposed completely in the K portion. The WT portion is essentially formed by at least one heat exchanger. In the WT portion a first fluid (heat exchange medium, stream of matter) flows in a closed conduit or duct system, and a second fluid (heat exchange medium, stream of matter) flows in the K portion, where, in the K portion, the energy input from the WT portion, or the heat exchanger thereof, results in at least partial evaporation of the fluid present in the K portion.

The evaporators are advantageously series-connected in two respects, as described below:
a) from the postreactor for a (feed) conduit into the K portion of the first evaporator, the WT portion of the first evaporator is operated with a heating medium,
b) the (first) tops outlet in the K portion of the first evaporator is connected via a conduit to the inlet of the WT portion of the second evaporator,
c) the (bottoms) outlet of the first evaporator is connected to the bottoms inlet of the second evaporator,
d) the outlet of the WT portion of the second evaporator is connected to the conduit leading to the (collection) tank, incorporating the heat exchanger, where the (tops) conduit from the second evaporator likewise flows into the conduit leading to the (collection) tank, and
e) the (bottoms) outlet of the second evaporator is connected as (feed) conduit to the first separation column via a conduit.

Advantageously, the following pressure control units are provided for establishment and delimitation of different pressure levels:
in the (bottoms) conduit from the (bottoms) outlet of the first evaporator to the (bottoms) inlet of the second evaporator and
in the conduit leading from the outlet of the WT portion of the second evaporator to the (collection) tank, in particular upstream of the inlet of the (tops) conduit of the second evaporator into that conduit and upstream of the heat exchanger therein.

In an advantageous process regime, the first separation column is operated at a lower pressure level different from the second evaporator, and so the plant advantageously likewise has a pressure control unit in the (bottoms) conduit from the (bottoms) outlet of the second evaporator to the inlet into the first separation column. In this variant of the process regime, the first separation column is operated at a pressure level 3 to 10 bar lower, ideally at a pressure level 3.5 to 5.5 bar lower.

Thus, in an advantageous embodiment of the plant, it may be the case that the WT portion of the first evaporator is connected to a media conduit, and where the conduit is guided between the evaporators such that the PACM-rich stream of matter is guided as a high boiler portion in the bottoms of the two evaporators.

In this case, in an analogous manner, the solvent-rich stream, which is then fed, for example, to a (collection) tank and/or directed back into the conditioning unit, is formed from the low-boiling (vapour) phase and at least partly condensed.

In one embodiment of the plant, it may thus be advantageous when a (material) conduit for the solvent-rich stream of matter leads from the tops connection of the K portion of the first evaporator to the inlet of the WT portion of the second evaporator, and wherein a conduit leads from the outlet of the WT portion to
a (collection) vessel and/or
a connecting unit/node with/into the (return) conduit for the solvent.

The solvent-conducting (return) conduit is connected to the conditioning unit and/or to at least one suitable collection tank.

The invention further encompasses a process for continuous catalytic hydrogenation of methylenedianiline (MDA; reactant1), especially 4,4′-diaminodiphenylmethane, with a hydrogen donor (reactant2), in particular a gaseous hydrogen donor, preferably hydrogen (H2), wherein production is implemented by means of an industrial plant. According to the invention, the plant is designed according to at least one of the above embodiments or variants.

In a preferred embodiment of the process, it may be the case that continuous catalytic production of methylenebis(cyclohexylamine) is effected, especially production of 4,4′-diaminodicyclohexylmethane (PACM), preferably 4,4′-diaminodicyclohexylmethane (PACM) with low proportions of trans/trans isomers. In a preferred process variant, continuous catalytic production of methylenebis(cyclohexylamine) is effected, especially production of PACM, of the formula (I)

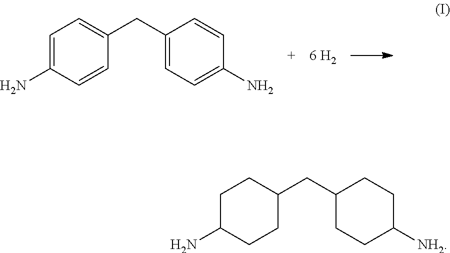 [SMILES: Nc1ccc(Cc2ccc(N)cc2)cc1 ; NC1CCC(CC2CCC(N)CC2)CC1]

The solvent is advantageously from the following group of substances: cyclohexane, dioxane, tetrahydrofuran (THF), cyclohexylamine, dicyclohexylamine, methanol, ethanol, isopropanol, n-butanol, 2-butanol, 2-methoxy-2-methylpropane (MTBE) or methylcyclohexane or a mixture thereof. In this case, an advantageous embodiment may be that the solvent is present in a proportion by weight of 40% to 50% by weight based on MDA in the reactant mixture.

In a further advantageous embodiment of the process, it may be the case that the at least one main reactor is operated at a pressure in the range from 60 bar to 120 bar, ideally in the range from 70 to 110 bar. In a further advantageous process regime, it may be the case that the pressure in the main reactor is 70 to 100 bar, ideally 80 to 90 bar. Particular preference is given to a pressure of about 85 to 90 bar. Advantageously, the at least one main reactor is operated at a temperature of 90 to 140° C., ideally 95 to 135° C. Even though the at least one main reactor is referred to in the present context as being “isothermal”, this ideal state is only achieved to a limited degree in industrial use, and so a temperature gradient of about 5 to 10° C. in radial direction and also in flow direction develops within the main reactor owing to incomplete heat dissipation.

In a further embodiment of the process, a further advantage may be that, from time to, the start of the process after renewal or regeneration of the catalyst, to time t4, the end of the process determined by renewal or regeneration of the catalyst, the operating temperature of the main reactor is increased and the temperature in the stream of matter in the inlet (feed) to the postreactor is maintained or lowered, where the increasing or lowering of the temperature is linear and/or stepwise.

In a further advantageous embodiment of the process, it may be the case that the pressure in the main reactor is 70 to 100 bar, ideally 80 to 90 bar.

In a further advantageous embodiment of the process, it may be the case that
the temperature at the main reactor inlet corresponds essentially to the temperature at the postreactor inlet, where essentially means a range or difference of +/−10° C. and/or the pressure at the main reactor inlet corresponds essentially to the pressure at the postreactor inlet, where essentially means a range or difference of +/−5 bar.

In a further advantageous embodiment of the process, it may be the case that the MDA (reactant1) comprises a mixture of the at least two isomers: 4,4′ MDA, 2,4′ MDA and 2,2′ MDA.

Ideally, the high proportion of trans/trans PACM in the product is in the range from 15% to 30% by weight, ideally 16% to 25% by weight.

In a further embodiment of the process, a further advantage may be that the MDA (reactant1) comprises a mixture of the following monomers: 4,4′ MDA, 2,4′ MDA and 2,2′ MDA, where the proportion of 4,4′ MDA is advantageously in the range from 75 to 98 mol %, ideally 85 to 95 mol %, preferably 90 mol %. The proportion of 2,4′ MDA in the reactant mixture is advantageously 7 to 15 mol %, preferably 8 to 12 mol %, ideally 9 to 10 mol %.

In a further advantageous embodiment of the process, it may be the case that the first separation stage of the separation unit to the (first) separation tank comprises at least one further (second) separation tank disposed downstream of and connected in series to the first separation tank. In this case, a bottoms outlet of the first separation tank is connected to an inlet of the second separation tank, where the first separation tank is operated at a first temperature and a first pressure and the second separation tank is operated at a second temperature lower than the first temperature (20° C. to 40° C. lower) and a lower pressure compared to the first pressure (1.5 bar to 4.5 bar lower). It is especially advantageous when an energy interconnection is undertaken in such a way that the stream of matter from the tops conduit (vapour conduit) of the first separation tank serves as heat source for the bottoms circulation system of the second separation tank. This energy interconnection can ideally be effected in a single heat exchanger or in two heat exchangers coupled via at least one media conduit.

Overall, all aspects, advantages and executions relating to plants or mentioned in association with the description thereof are also intended to be applicable identically or analogously to the process, and vice versa, unless stated otherwise and/or there is a technical impossibility associated with analogous application.

The solution according to the invention is described in detail hereinafter with reference to working examples.

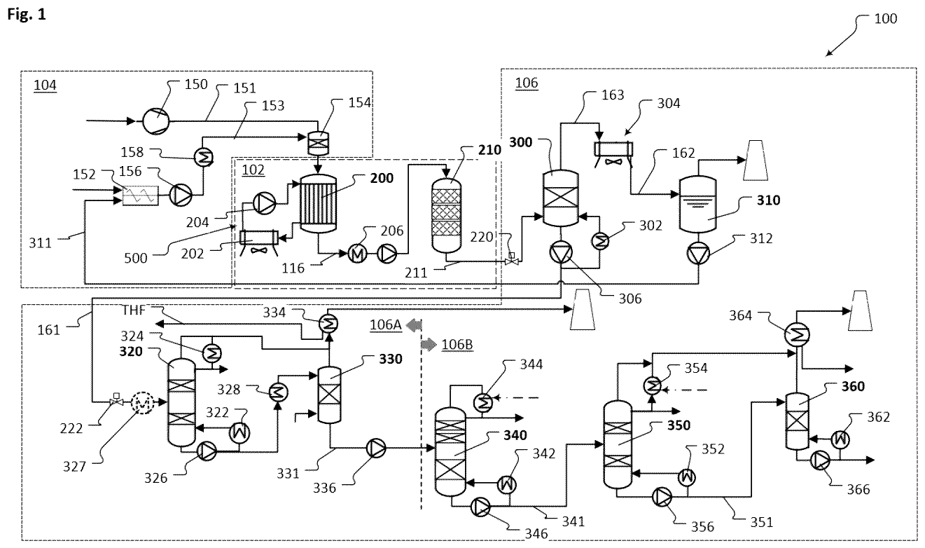

FIG. 1 shows the plant 100 for continuous production of 4,4′-diaminodicyclohexylmethane (PACM) by catalytic hydrogenation of methylenedianiline (MDA; reactant1) with a hydrogen donor (reactant2), where the hydrogen donor is supplied in the form of gaseous hydrogen (H2). The plant 100 comprises a conditioning unit 104 for the reactants, a reactor unit 102 and a separation unit 106.

The conditioning unit 104 is framed by dashed lines and comprises (feed) conduits for the reactant1 and the hydrogen (reactant2), and also the solvent. In addition, the conditioning unit comprises a compressor unit 150, referred to hereinafter as compressor in the conduit 151 supplying the hydrogen, a mixer 152 in the conduit supplying the solvent and reactant1. Additionally disposed in the conduit 153 supplying the mixture of reactant1 and solvent are a pump 156 and a heat exchanger 158. The conduits 151, 152 open into a mixing vessel 154 disposed upstream of the main reactor 200. The mixing vessel 154 serves for intensive mixing of the reactants, and its outlet forms the inlet for the main reactor 200.

The reactor unit 102 is framed by dashed lines and comprises essentially the main reactor 200, a cooling circuit 500, a postreactor 210 and a heat exchanger 206 in the feed conduit to the postreactor 210. The cooling circuit 500 incorporates a pump 204 and a heat exchanger 202, where the circulating coolant in the main reactor 200 flows around the catalyst material-filled carrier elements. In the example shown, the flow direction toward the catalyst material-filled carrier elements is in cocurrent direction. The postreactor 210 is connected via the conduit 211 to the separation unit 106, i.e. the first separation stage thereof.

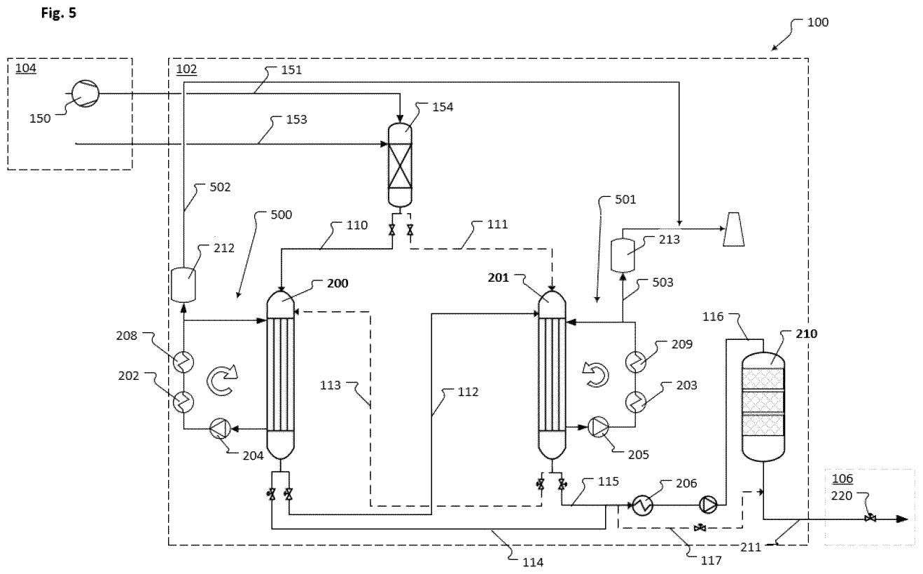

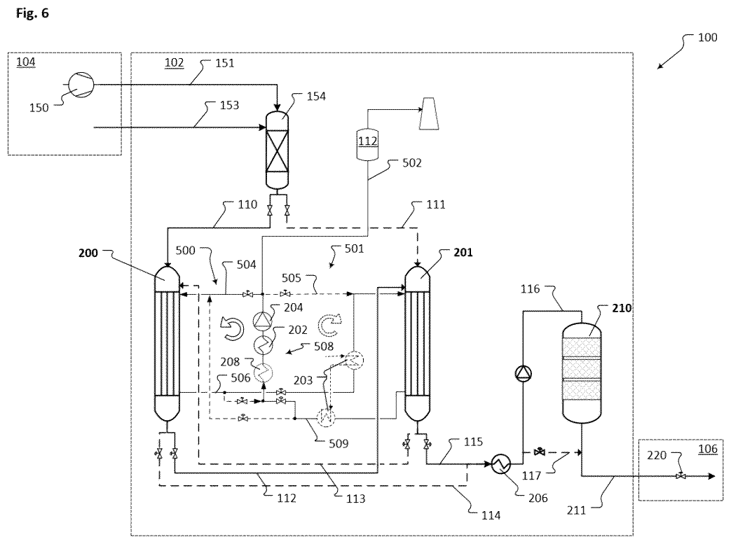

The heat exchanger 202 in the cooling circuit 500 is shown as an air-cooled heat exchanger 202, but may also be alternatively designed in order to control the temperature of the cooling medium in the cooling circuit 500 in indirect heat exchange, for example by means of a flowing cooling medium, such as an oil, water or a brine. The cooling circuit 500, in a plant variant which is not shown, also incorporates a heat exchanger, analogously to the heat exchangers 208, 209 in FIGS. 5 and 6. The latter is operated with a heating medium and serves, in the step of starting up the main reactor 200, to adjust the temperature of the main reactor 200 to about 80 to 100° C., ideally to a temperature of 85° C. to 95° C. In the plant and process example shown, in which one aim is to achieve a minimum trans/trans isomer ratio of about 17% to 23% by weight, the main reactor 200 filled with fresh or regenerated catalyst is preheated to a temperature of about 90° C. by means of the heat exchanger 208. The main reactor 200 is operated at a pressure of 87 to 88 bar.

The separation unit 106 is framed by dashed lines and comprises a plurality of separation apparatuses for separating the solvent, especially in a first separation stage, and the PACM product, especially in a second separation stage 106B, from the rest of the reactant and by-products. The first separation stage (not displayed) comprises a separation tank 300 (flash vessel) to which a bottoms circulation system is connected, incorporating a heat exchanger 302 and a pump 306. The top outlet of the separation tank 300 is connected to a heat exchanger 304, a condenser, downstream of which is provided a collecting vessel 310 for the solvent. By means of the condenser 304, about 80% of the solvent, THF in this case, is condensed out and could be collected or returned. In the flash stage, an energy requirement of about 1400 KW is required for the heat exchanger 302 in the bottoms circulation system of the separation tank 300.

The condensation unit 304 is shown as a heat exchanger in the design of an air-cooled apparatus, but may also alternatively be designed to at least partly condense and to cool the vaporous stream of matter at the outset in indirect heat exchange, for example by means of a flowing cooling medium such as a cooling water or a medium suitable for thermal integration.

The amounts of energy specified herein are calculated for a plant output of the PACM product in the synthesis reaction mentioned of about 3.37 t/h, and a by-product output of about 0.4 t/h of HB and about 0.05 t/h of LB. The ratio of the mass flow rates of THF to MDA was 4.2 to 4.5.

In addition, the first separation stage of the separation unit 106 for further removal of the solvent comprises a first separation column 320 and a second separation column 330, where the second separation column 330 takes the form of a stripping column. For this purpose, nitrogen (N2) is advantageously used as a stripping medium, which is passed through the column in countercurrent to the stream of matter. By means of the pump 306 disposed in the bottoms outlet of the separation tank 300, the solvent-depleted stream of matter can be diverted via the conduit 161 to the first separation column 320. An expansion unit 222 is provided in the conduit 161 and is designed as a controllable valve in the example shown. The stream of matter is introduced into the first separation column 320 via a central inlet as shown. Additionally disposed upstream of the first separation column 320 is a (forward) heat exchanger 327 (shown by dashed lines), which constitutes an option for heating of the first separation column 320.

In the first separation column 320, the solvent concentration is reduced from 30% by weight in the feed via the product-rich stream of matter from the conduit 161 to about 2% by weight of residual solvent.

The first separation column 320, equipped with (structured) packings, is connected to a bottoms circulation system incorporating a heat exchanger 322 and a pump 326. In addition, a tops circulation system is disposed at the column top of the first separation column 320, incorporating a heat exchanger 324 designed as a condenser. Two outlets for the solvent-rich stream of matter lead out of the tops circulation system, with one of the two outlets leading into the tops outlet of the downstream separation column 330 (stripping column).

In particular, the solvent separated off in the first separation stage is conductable via the conduit 311 into a collection tank (not shown) and/or the mixer 152 of the conditioning unit 104. In the conduit 211 leading from the postreactor 210 to the separation tank 300, there is a pressure control unit 220, designed in the present example as a controllable valve.

The product-rich stream of matter having a residual content of about 2% by weight of solvent (THF) is directed from the bottoms outlet of the first separation column 320 via the conduit 321 into the top of the second column 330, the stripping column. This conduit 321 incorporates a heat exchanger 328. The second column 330 has at least one internal packing, where a gas feed for an inert gas (stripping gas) in particular is disposed below the packing, such that the introduced product-rich stream of matter flows through the second column 330 in countercurrent principle to the stripping gas and is further depleted of solvent. In the plant example shown, nitrogen (N2) is used as stripping gas. The solvent-rich vapour from the second separation column 330 is directed via the tops outlet into a condenser 334, together with the vapour coming from the tops outlet of the first separation column 320. The solvent stream condensed out in the heat exchanger 334 is returned to the conditioning unit 104, wherein the uncondensable portion is led off from the heat exchanger 334 and, for example, fully thermally oxidized.

The product-rich stream of matter is fed from the bottoms outlet of the second separation column 330 to the third separation column 340 via the conduit 331, incorporating the pump 336. The second separation stage 106B of the separation unit 106 serves in particular to separate the LB and/or HB by-products from the PACM product.

The second separation stage comprises essentially three separation columns, where the third separation column 340, the first of the second separation stage, is supplied centrally with the stream of matter. The third separation column is connected to a bottoms circulation system incorporating a heat exchanger 342 and a pump 346. The product-rich stream of matter is directed via the conduit 341 from the bottoms outflow to the fourth separation column 350. In addition, the third separation column 340 is connected to a tops circulation system incorporating a heat exchanger 344. Condensed LB is led off as the first by-product from this tops circulation system.

The product-rich stream of matter is fed centrally to the fourth separation column 350 via the conduit 341. The fourth separation column 350 is connected to a bottoms circulation system incorporating a heat exchanger 352 and a pump 356. In addition, the fourth separation column 350 is connected to a tops circulation system incorporating a heat exchanger 354 in the form of a condenser. The product-rich stream of matter is discharged from the tops circulation system as condensate via the tops outlet. In the present case, according to the example shown, the uncondensed vapour stream and/or gas stream is introduced into the tops discharge of the downstream fifth separation column 360. Subsequently, product is condensed further and discharged via a further heat exchanger 364 (condenser). The stream of matter with a low level of product is discharged via the bottoms outlet of the fourth separation column 350 via the conduit 351 and introduced into the top portion of the fifth separation column 360. This stream of matter is highly enriched with HB, a second by-product. The fifth separation column 360 is connected to a bottoms circulation system incorporating a heat exchanger 362 and a pump 366. Furthermore, the separation column 360 has a tops outlet which leads to the aforementioned heat exchanger 364 in the form of a condenser. In this condenser 364, a further product-rich stream of matter is condensed out as condensate and discharged, with gaseous discharge of the uncondensable portion. In particular, the latter may subsequently be fully oxidized.

With regard to the stream of matter, the reactor unit 102 is operated at a first, high pressure level of about 60 to 120 bar, the first separation stage of the separation unit 106 is operated at a second, low pressure level of 4 to 12 bar, and the first separation column 320 and the second separation column 330 are operated at a third, slightly elevated pressure level of 1.05 to 2.5 bar. In the example shown, the first pressure level is 80 to 90 bar, the second pressure level is 4.5 to 7.5 bar and the third pressure level is 1.1 to 1.2 bar.

In addition, the reactor unit 102, with regard to the stream of matter, is operated at a first, high temperature level of about 90° C. to 140° C., where the first separation stage of the separation unit 106 is operated at a second, low pressure level of 4 to 12 bar, and the first separation column 320 and the second separation column 330 are operated at a third, slightly elevated pressure level of 1.05 to 2.5 bar. In the example shown, the first pressure level is 80 to 90 bar, the second pressure level is 4.5 to 7.5 bar and the third pressure level is 1.1 to 1.2 bar.

With direct temperature control of the main reactor 200 via the cooling circuit 500 in series with the uncooled postreactor 210, surprisingly, a significantly reduced energy requirement compared to the prior art and a simplified, more stable process regime has been demonstrated. Without being tied to any specific interpretation, this success is apparent in that the cooling circuit 500 only has to be specifically designed for the very vigorous initial reaction for dissipation of the exothermic heat of reaction, while the still significant postreaction has to be adjusted via the feed temperature by means of the upstream heat exchanger 206 alone. Because the reaction in the postreactor 210 is already highly attenuated, the stream of matter is only heated by about 5 to 15° C. and can be discharged into the separation tank at this level without any problems.

All in all, the figures show internals such as packings, separation planes, support elements etc. in the apparatuses such as reactors, separation columns, vessels etc. by means of the corresponding symbols. These each indicate advantageous embodiments, with regard to the number, type and/or relative position to the respective feed conduit or discharge conduit. For example, the illustration of first separation column 320 thus indicates that, advantageously, the (feed) stream of matter is introduced via the conduit 161 in such a way that at least one (theoretical) separation plane is present in each case between the tops circulation system and the bottoms circulation system. The determination of the specific type and/or number of separation planes is known to the skilled person and can be varied or provided for in an appropriate manner.

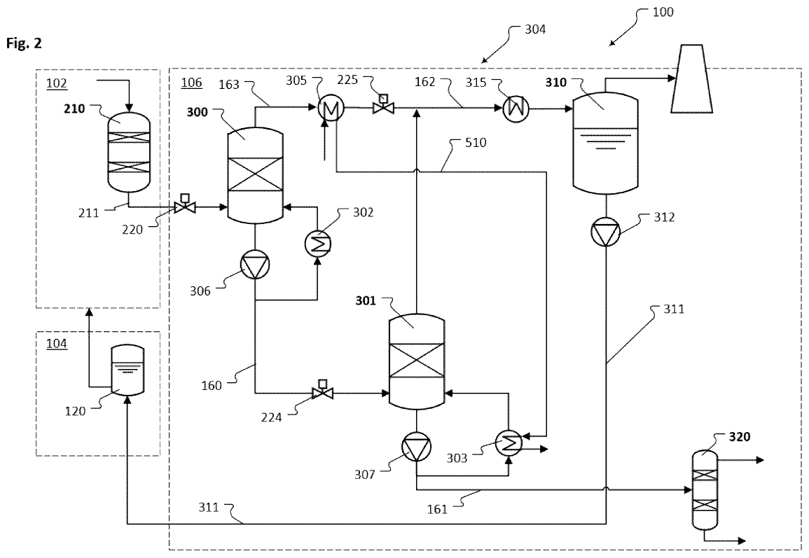

FIG. 2 shows a first embodiment of a two-stage expansion with advantageous interconnection of the heat exchangers in the first separation stage of the separation unit 106. By contrast with the variant shown in FIG. 1, a further (second) separation tank 301 is disposed downstream of the (first) separation tank 300. The further separation tank 301 likewise has a bottoms circulation system incorporating a heat exchanger 303 and a pump 307. The bottoms outlet of the first separation tank 300 is connected to the bottoms inlet of the second separation tank 301 via the conduit 160, and the bottoms outlet of the second separation tank 301 is connected via the conduit 161 to the first separation column 320 in an analogous manner to FIG. 1. The two separation tanks 300, 301 are operated at slightly different pressure levels, for which a pressure control unit 224 is arranged in the connecting conduit 160 between the two separation tanks 300, 301, which is designed in the present example as a valve controllable by open-loop and closed-loop control. The product-rich stream of matter is fed to the first separation column 320 via the bottoms conduits 211, 160, 161 via the two separation tanks 300, 301. The solvent-rich vapour or condensate stream is fed via the respective tops outlets of the two separation tanks 300, 301 into the conduit 162 and introduced into the collection vessel 310, wherein the conduit 162 incorporates two heat exchangers 305, 315 designed as condensers. The first section of the conduit 162 from the tops outlet of the separation tank 300 to the heat exchanger 304 or 305 is indicated by reference numeral 163.

FIG. 2 shows the heat exchangers 305, 303 as two separate apparatuses, where the solvent (THF) is condensed at high pressure in the heat exchanger 305 (condenser) and the enthalpy of condensation released in the heat exchanger tubes is used in the heat exchanger 303 to evaporate further solvent at lower pressure in the separation tank 301. The EC of the two heat exchangers 305, 303, in an embodiment variant which is not shown in FIG. 2, may also be implemented as an integrated media-based EC in a single apparatus.

The pressure control unit 224 in the connecting (bottoms) conduit 160 splits the second pressure level of 4.5 to 7.5 bar in that the first separation tank 300 or the conduit 160 upstream of the pressure control unit 224 is operated at a pressure elevated by about 1.5 to 2.5 bar compared to the second separation tank 301 or the pressure level in the conduit 161 upstream of the pressure control unit 222. Thus, in the present case, the pressure level in the conduit 160 is at the level of 3.5 to 5.5 bar, especially about 4.3 bar, where the second separation tank 301 or the conduit 161 upstream of the pressure control unit 222 is operated at a pressure level of about 1.0 to 2.0 bar, especially about 1.3 bar. In addition, a pressure control unit 225 is provided in the (steam) conduit 162 upstream of the inlet coming from the tops outlet of the second separation tank 301, such that, analogously, two pressure and temperature levels can also be established in the vapour conduit 162, 163. In the example shown, the steam leaves the first separation tank 300 at about 4.0 to 4.5 bar and a temperature of about 110 to 120° C., and the steam in the second separation tank 301 is released at about 1.1 to 1.5 bar and a temperature of about 85 to 95° C. and introduced into the conduit 162.

With regard to the media flow and the energy requirement, the feed of matter via the conduit 211 is at a temperature of about 115 to 130° C. and the bottoms circulation system is operated at a temperature elevated by about 10° C. for this purpose, where the heat exchanger is operated in the bottoms circulation system of the first separation tank 300 with steam as (heat exchange) medium. The (cooling) medium fed to the first condenser 305, for example water, is heated and supplied as (heating) medium via the conduit 510 to the heat exchanger 303 incorporated in the bottoms circuit of the second separation tank 301. By comparison with the single-stage flash unit and condensation by means of one condenser 304, as shown in FIG. 1, it was thus possible for the energy requirement for the heat exchange in the bottoms circulation system of the separation tanks 300, 301 to be lowered by about 50%, from previously about 1440 kW for the heat exchanger 302 to about 700 kW remaining for the heat exchanger 302 of the first separation tank 300. In addition, it was possible for the volume of the first separation tank 300 to be designed to be about 20% smaller, and the electrical pump conduit of the bottoms pump 302 to be reduced by about 15%.

In addition to the bottoms outlet, the collection vessel 310 has a tops outlet via which the uncondensed gas fraction can be led off, especially led off to a complete thermal oxidation treatment unit.

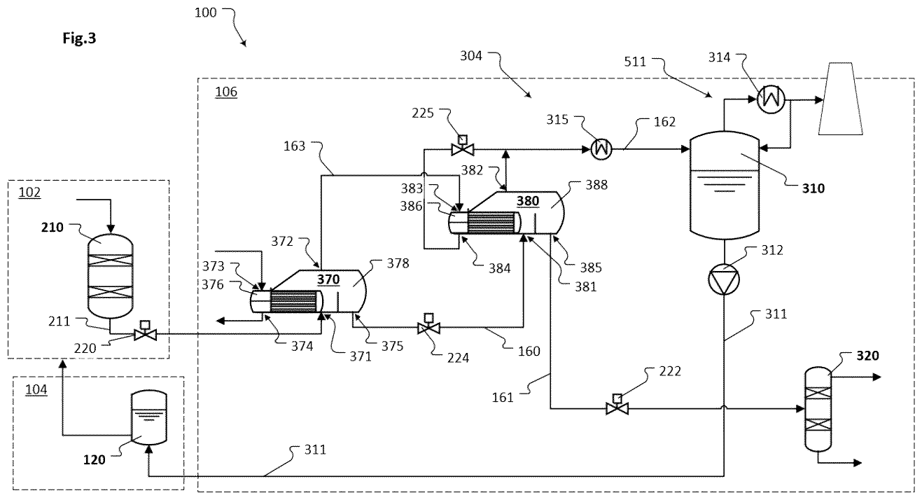

FIG. 3 shows another embodiment of a two-stage expansion with a particularly advantageous interconnection in the first separation stage of the separation unit 106, where the EC is implemented as a series EC. In this case, instead of the separation tanks, two evaporators 370, 380 are arranged in series or interconnected crosswise, as outlined below. The evaporators 370, 380 are so-called kettle-type evaporators, which have a heat exchanger portion 376, 386 (WT portion) closed to the flowing fluid and a tank portion 378, 388 (K portion) open to the flowing fluid. The WT portions 376, 386 each have an inlet 373, 383 and an outlet 374, 384, where the fluid is guided in closed channels or pipes, for example at least one shell-and-tube system. The K portions 378, 388 each have at least one (bottoms) inlet 371, 381, each have one (bottoms) outlet 375, 385 and each have at least one (tops) outlet 372, 382, where the fluid introduced via the at least one (bottoms) inlet in particular is heated and at least partly evaporated by means of the respective WT portion or the associated heat exchanger. The respective tank portion may be in two-part form, as in the example shown. In this case, the (bottoms) inlet is disposed in a central K portion, into which the heat exchanger of the WT portion also projects and in which the energy is introduced into the K portion. The (bottoms) outlet is disposed in a lateral or outer K portion, in which it is advantageous, but not obligatory, for internals to form a calmed zone for the liquid mixture of matter and/or into which the heat exchanger of the WT portion does not project.

In the example shown, the WT portion of the first evaporator 370 is charged via the inlet 373 with steam at a temperature of about 120 to 140° C. at a pressure of about 3 bar. Via the (bottoms) inlet 371, the stream of matter from the postreactor 310 enters the K portion 378, where it is partly evaporated. The solvent-rich vapour fraction is directed from the (tops) outlet 372 via the conduit 163 as heating medium to the WT portion 386 into the inlet 383 of the second evaporator 380, and introduced into the collection tank 310 from the outlet 384 via the condenser 315, the condensation therein, and the conduit 162. Compared to the embodiment according to FIG. 2, in the cascade composed of the two evaporators 370, 380 shown, the condenser 305 and the (bottoms) heat exchanger 303 are combined in one apparatus or in one heat exchanger, namely the WT portion 386 of the evaporator 380. The vapour fraction from the second evaporator 380 is introduced analogously to the working example of FIG. 2 into the conduit 162, upstream of the condenser 315. The (bottoms) outlet 385 is connected to the first separation column 320 via the conduit 161. Likewise in an analogous manner, the two evaporators 370, 380 are operated at different pressure and temperature levels, which essentially correspond to those of the variants according to FIG. 2, and so a pressure control unit 224 is disposed in the bottoms conduit 160 from the outlet 375 of the first evaporator 370 to the inlet 381 of the second evaporator 380.

The advantage of this embodiment is a more compact design with lower area and space requirements. Furthermore, because of the theoretical integration of the two heat exchangers 305, 303 in one internal W portion 386 of the second evaporator 380, lower heat losses occur as a result of the lower conduit lengths, here in particular the omission of the conduit 510. For the same reason, there is a reduction in expenditure on insulation of conduits/pathways.

In the embodiment of FIG. 3 shown, the collection vessel is connected to a tops circuit 511 incorporating a heat exchanger 314 designed as a condenser. This can be used to separate off further solvent (THF).

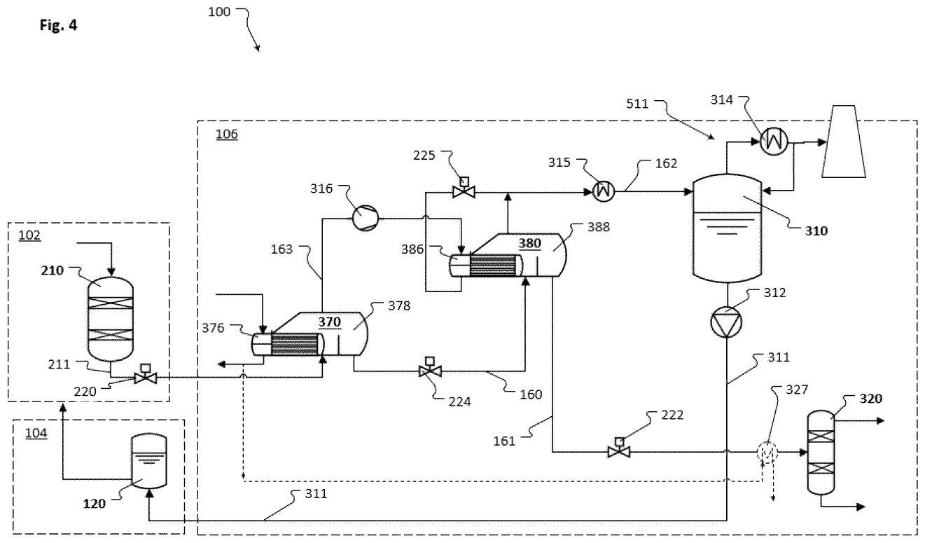

In a further-improved embodiment, as shown in FIG. 4, a portion of the energy requirement is introduced by electrical energy in that a compressor 390 is provided in the conduit 163. As a result, the temperature raises the energy level in the stream of matter and, if necessary, liquefies a portion, so as to elevate efficiency in the WT portion 386.

In addition to the benefit of elevated efficiency, another advantage is that the volume flow rate in the conduit 163 is greatly reduced by the partial liquefaction, such that the downstream components, especially of the WT portion 386, may be of smaller dimensions or more heat exchange area in the interior of the K portion 388 can be provided in the same installation space, so that the evaporator 380 overall has higher efficiency.

FIG. 4 shows a further-optional embodiment which can likewise be provided in FIG. 3. A (forward) heat exchanger 327 is provided upstream of the first separation column 320. Advantageously, the outflowing media stream from the WT portion 376 of the first evaporator 370, at a temperature of about 105 to 125° C., is supplied to the EC as heating medium to this (forward) heat exchanger 327. In this way, the energy requirement of the heat exchanger 322 in the bottoms circulation system of the first separation column 320 is reduced in a virtually linear manner. In an embodiment which is not shown, an integrated material-based EC is provided in that the heat exchangers 376 and 327 are designed integrated as structurally a (single) apparatus.

FIG. 5 shows an improved variant of the reactor unit 102. Two main reactors 200, 201 here are switchably connected to one another in series. Some of the valves/valve units that can be controlled by open-and/or closed-loop control are shown in FIG. 5; others can be provided if required by the person skilled in the art in order to ensure safe operation of the two main reactors 200, 201. The two main reactors 200, 201 are each incorporated into a cooling circuit 500, 501, with which the fixed bed reactor 200, 201 is kept in each case at a permissible, cooled reaction temperature. In the interconnection of the main reactors 200, 201 illustrated and shown with solid lines, the flow passes first through the first main reactor 200 via the conduit 110 coming from the mixing vessel 154 and the first part of the reaction takes place in this main reactor 200. Downstream via a lower outlet and the conduit 112, the mixture of matter is fed into the top of the second main reactor 201 and leaves it via a bottoms outlet and conduit 115 into the common conduit 116 as a feed into the common postreactor 210. The common conduit 116 incorporates a heat exchanger 206, by means of which the temperature level required for the postreactor 210 can be ensured. The sequence of the flow through the two main reactors can also be reversed, from the main reactor 201 to the main reactor 200. For this purpose, in an analogous manner, the flow to the main reactor 201 first passes through the conduit 111, with the conduit 110 to the top of the main reactor 200 closed. The mixture of matter leaves this fixed bed reactor via a bottoms outlet and the (intermediate) conduit 113 which is connected to the top of the main reactor 200, with the conduit 115 closed. Finally, the mixture of matter leaves the main reactor 200 via a bottoms outlet and conduit 114, which opens analogously into the common conduit 116 and thus leads to the postreactor 210. Each of the two cooling circuits 500, 501 has a conduit 502, 503 which branches off and is guided in each case through a container 212, 213, which serve as pressure equalization vessels, and further to a chimney and/or a complete oxidation unit. The conduits and valves/valve units are present and designed in such a way that each of the main reactors 200, 201 can be operated alone and the stream of matter can completely bypass the respectively other main reactor. The flow direction of the two cooling circuits 500, 501 is symbolized by an arrow, where the two cooling circuits 500, 501 are advantageously identical or substantially identical, since the two main reactors 200, 201 are operated alternately with respect to the flow direction of the reactant stream or the stream of matter as the first or second main reactor.

However, the cooling circuits 500, 501 are designed and controllable in such a way that, depending on the process-related requirements, such as, in particular, product management and/or product quality, autonomous cooling outputs or cooling functions are achieved in each case. This relates in particular to the volume flow per unit time of cooling medium and/or the temperature level or the permissible heating of the respective cooling medium. Furthermore, there is a direct option for thermal integration of the cooling circuits 500, 501 with other plant sections or heat exchangers, where the heat exchangers 202, 203 serve as heat source by receiving the heat of reaction, such that the total energy consumption of the plant or the process for producing PACM can be lowered.

FIG. 5 also shows an optional conduit 117 as a dashed line (bypass 2), by which the postreactor 210 can be bypassed if, for example, it has to be maintained and/or the catalyst charge has to be renewed. In this case, the temperature at least of the second main reactor in flow direction of the stream of matter is controlled such that complete reaction is ensured with the desired product quality, in particular the desired proportion of the respective isomers. The conduit branch of the conduit 117 may be provided upstream or downstream of heat exchanger 206 in flow direction, advantageously upstream of heat exchanger 206, in order also to be able to bypass it if necessary, and to be able to undertake necessary maintenance operations while the plant is running.

The main reactor 200 can be bypassed in operation of the main reactor 201 shown on the right in the picture via the conduits 111, 115 and 116. The main reactor 201 can be bypassed in operation of the main reactor 200 shown on the left in the picture via the conduits 110, 114 and 116, such that there is no flow through the (cross-) conduits 112, 113 between the two main reactors 200, 201 in each case.

The cooling circuits 500, 501 also (optionally) incorporate heat exchangers 208, 209 in the plant variant shown. These are operated with a heating medium, especially steam, and serve for (pre) heating the reaction temperature of the respective main reactors 200, 201 in the startup step of the main reactors 200. In the plant and process example shown, as already set out for FIG. 1, with a view to the aim of achieving a minimum trans/trans isomer ratio of about 17% to 23% by weight, it is advantageous when the newly catalyst-filled main reactor 200, 201 is preheated to a temperature of about 90° C. by means of the respective heat exchanger 208, 209, or the respective newly filled main reactor 200, 201 is correspondingly preheated.

The preheating to typically 85 to 95° C. makes it possible for the reaction in the case of the present catalyst to start immediately, without or substantially without recycling streams of the mixture of matter from the process until the desired reaction temperature has been attained.

The particular advantage of the switchable main reactor 200, 201 is the possibility of operating the main reactor, through which the flow passes first, at a higher temperature because the catalyst is already partly exhausted and inactivated, essentially without adversely affecting (i.e. increasing) the trans/trans isomer ratio. At the same time, more significantly heated cooling medium is obtained in the respective (circulation) heat exchanger 202, 203 of the main reactor 200, 201 through which the flow passes first. Because of the higher temperature, this hotter heat exchange medium can be better used in the plant for integrated media-based EC and/or for customary heat exchange with a reactant stream of matter.

The variant of the switchable main reactors 200, 201 as shown in FIG. 6 differs from that according to FIG. 5 in that the cooling circuit 500 of the first main reactor 200 is likewise connected in series with the cooling circuit of the second main reactor 201. In this case, only one (cooling) heat exchanger 202 and only one pump 204 is provided for the common cooling and the media circuit, and so the flow through the common (central) conduit branch 508 in both cooling circuits 500, 501 is generally constant and only in one direction, irrespective of the interconnection of the two main reactors 200, 201. Of course, the “central” conduit branch need not in fact be disposed between the two main reactors 200, 201. The variant is shown as solid lines, in which the first main reactor 200 (on the left) is first fed with the reactant mixture via the conduit 110 and also the introduction (of cooling media) downstream of the heat exchanger 202 and the pump 204 is first effected via this main reactor 200. The conduits that do not carry media in this circuit or conduits that carry the mixture of matter are shown by dashed lines. In these variants too, it is possible to completely bypass the respectively other main reactor with the mixture of matter and/or the (cooling) medium. For example, in the process of filling one of the two main reactors 200, 201, the respectively other main reactor can thus continue to be operated at maximum output. The bypass of the stream of matter or of the respective main reactor 200, 201 is analogous to FIG. 5.

In the example circuit shown, the flow passes through the central conduit branch 508 and the heat exchanger 202 and is directed via the conduit 504 in cocurrent into the media space of first main reactor 200, blocking conduit 505 that leads to the common conduit node coming from the second main reactor 201. The medium leaves the first main reactor 202 via the conduit 506 at a low-level outlet and is directed to a high-level inlet into the media space of the second reactor 201 (cross-conduit). The medium likewise flows through the media space of the second main reactor 201 in cocurrent and leaves it at a low-level outlet via the conduit 509, from which a branch again flows into the central conduit branch 508, and so flow through the circuit can continue. Similarly, the flow passes firstly via the conduit 505 through the main reactor 201 shown on the right when the conduit 504 is blocked. The medium then leaves the media space of the second main reactor 201 via the conduit 509 and is directed into the media space of the other main reactor 200 at a high-level inlet. The outlet of the media space at a low-level point leads into the conduit 506 and thence via a branch into the central conduit branch 508.

FIG. 6, analogously to FIG. 5, shows the optional conduit 117 as a dashed line (bypass 2), which allows the postreactor 210 to be bypassed, where the conduit 117 branches off downstream of the (forward) heat exchanger 206 of the postreactor 210.

FIG. 6 also shows a plant variant (dashed line) whereby higher control security or a higher degree of freedom of temperature management is obtained in the respective (cross-) conduit 504, 505 of the cooling circuits 500, 501 in that switchable and controllable heat exchangers 203 (feed coolers) are arranged as required. In the working example shown, the two heat exchangers 202 for the EC are connected in series since, because of the respectively inactive (cross-) conduit, the conduit 505 in the example shown, no heating takes place at the site of installation for the heat exchanger 202. The cooling media conduit or the cooling media circuit of the heat exchangers 202 can be operated in an advantageous variant in a sidestream or via a secondary or auxiliary circuit via the pump 204.

The great advantage of this sidestream or secondary/auxiliary circuit of a coolant via the heat exchangers 203 is that this additional cooling output only needs to be called on as required and, with a low degree of complexity, a greater extent of closed-loop temperature control as required is possible in the respectively second of the two series-connected main reactors.

The (forward) heat exchanger 206 is described primarily in the present context and is in some cases shown in an EC in which it operates as a heat exchanger 206 primarily as a heat sink, meaning that the stream of matter conducted therein is heated. Because of the dependent mode of operation of the postreactor 210 with adaptation to the main reactor 200, 201, cooling of the stream of matter in the (feed) conduit 116 upstream of the postreactor 210 may be required at least temporarily, in particular permanently, because about 10% to 20% of the conversion occurs in the (adiabatic) postreactor, such that the stream of matter is heated up to about 140° C., measured in the outlet. Thus, independently of the embodiments and variants of the plants and of the process that are described herein, it is possible to provide an adapted EC for cooling (heat exchanger 206 operates as a heat source). Alternatively or additionally, supplementary cooling may be provided via a modified or additional EC.

Overall, a multitude of customary open-loop and closed-loop control elements that are known to the person skilled in the art and are necessary or advisable for an advantageous process regime are not shown, such as sensors (flow, temperature, pressure etc.), displays, setting and control elements (especially valves, further pumps, compressors), collecting vessels etc., and should be added if required. In particular, when “a” pump or “a” compressor is mentioned, this also means customary redundancies from at least two parallel aggregates, especially of two parallel pumps or two parallel compressors. In an analogous manner, “a heat exchanger” should not be understood to be limiting and, with the respective local heat exchange function, also means arrangements of heat exchangers that are connected in series or in parallel, including redundant heat exchangers, which in this context does not mean interconnections with at least one further heat exchanger and a locally different heat exchange function.

All heat exchanges and condensers are fundamentally designed such that indirect heat transfer occurs and there is no physical mixing of reactants, product, by-products and/or solvent with the (heating/cooling) medium, such as gas, steam, water, oil, brine etc., unless stated otherwise.

Even if components such as valves, pressure control unit, isolators etc. are shown individually or separately in the present context for simplified description and to some degree were not mentioned individually, this should not be read in a limiting manner; instead, the person skilled in the art is able to combine two or more of these components in a valve unit or control unit as required or provide multiway valves instead.

In the present case, what is meant by “upstream” or “downstream” is the arrangement and/or flow direction of the product-rich stream of matter, unless stated otherwise. Furthermore, “media”, “media stream”, “media conduit”, etc., always means a heating or cooling medium or the associated conduit, unless stated otherwise.

For all embodiments and variants of the plant and of the process, it may generally be the case that the second separation column 340 and the third separation column 350 are executed as a single column, in particular as a dividing wall column (not shown). In this case, the separation functions of the second separation column 340 and of the third separation column 350 can advantageously be performed at least partly, ideally completely, by means of the dividing wall column (not shown), as known, for example, from documents EP 012 62 88 B1 or EP 012 23 67 A2.

Overall, a large, energy benefit can be achieved with the plant according to the invention and the process, wherein a significant reduction in the externally supplied energy flows was enabled, in particular saving of large amounts of (external) heating steam.

1. A plant for continuous catalytic hydrogenation of methylenedianiline (MDA; reactant1) with a hydrogen donor (reactant2), the plant comprising:
a conditioning unit for the reactants,
a reactor unit, and
a separation unit,
wherein the conditioning unit comprises (feed) conduits for reactant1, reactant2 and at least one solvent, at least one heat exchanger in at least one (feed) conduit, at least one mixer for mixing the reactants and/or at least one reactant with at least one solvent;
wherein
the reactor unit comprises at least one fixed bed reactor as main reactor with an immobile catalyst packing, wherein the at least one main reactor comprises
a first flow pathway for a mixture of matter through the immobile catalyst packing and
another separate closed flow pathway for a heat exchange medium outside the immobile catalyst packing, and with a heat exchanger integrated into a media circulation;
the separation unit comprises at least:
a first separation stage for removal of the at least one solvent and
a second separation stage for separation of the at least one reactant and/or at least one by-product from a product,
wherein
the separation unit in the first separation stage comprises at least one pressure control unit, at least one separation tank and at least one condensation unit for the at least one solvent, where a (return) conduit for the at least one solvent leads from the at least one condensation unit of the first separation stage to the conditioning unit.

2. The plant according to claim 1, wherein the reactor unit comprises a first main reactor and at least one downstream permanent non-switchable further postreactor.

3. The plant according to claim 2, wherein the reactor unit comprises a second main reactor as a fixed bed reactor comprising
a first flow pathway for the mixture of matter and
a further (closed) flow pathway for a heat exchange medium, and where a valve unit is provided upstream of the first main reactor and the second main reactor in the at least one (feed) conduit, by which a volume flow of the reactant mixture is divisible, conductable and/or completely switchable between the first main reactor and the second main reactor, and where the first main reactor and the second main reactor are connected
each to one heat exchanger or
to a common heat exchanger for the further (closed) flow pathway.

4. The plant according to claim 3, wherein at least one heat exchanger is disposed in a conduit between the at least one main reactor and the postreactor.

5. The plant according to claim 1, wherein the separation unit comprises a first separation tank connected via a conduit to the at least one main reactor or the postreactor, where the separation tank comprises
a tops outlet,
a base/bottoms outlet, and
a heatable bottoms circulation system having at least one heat exchanger, where a (tops) conduit incorporates a condensation unit, and where the following are disposed downstream of the condensation unit:
a (collection) vessel and/or
a connecting unit/node to/into the (return) conduit for the at least one solvent.

6. The plant according to claim 5, wherein the separation unit comprises a second separation tank disposed downstream of the first separation tank, where the base/bottoms outlet of the first separation tank is connected to an inlet of the second separation tank, where the second separation tank comprises
a tops outlet,
a base/bottoms outlet, and
a heatable bottoms circulation system having at least one heat exchanger, where a (tops) conduit leads from the (tops) outlet of the second separation tank to a condensation unit and/or into the (tops) conduit of the first separation tank.

7. The plant according to claim 6, wherein the condensation unit comprises at least two heat exchangers, where a connecting unit/node for the (tops) conduit of the second separation tank to the (tops) conduit of the first separation tank is disposed between the at least two heat exchangers of the condensation unit.

8. The plant according to claim 6, wherein a (media) conduit from an outlet from the condensation unit leads to an inlet of the at least one heat exchanger of the second separation tank.

9. The plant according to claim 1, wherein the separation unit in the first separation stage comprises at least two series-connected evaporators as separation tank, where a condensation unit provided in a (tops) conduit of at least one of the two series-connected evaporators is at least one heat exchanger, and where the following is disposed in a (tops) conduit downstream of the at least one heat exchanger:
a (collection) vessel and/or
a connecting unit/node to/into the (return) conduit for the at least one solvent.

10. The plant according to claim 9, wherein at least one of the at least two series-connected evaporators or all series-connected evaporators take a form of what are called kettle-type evaporators.

11. The plant according to claim 9, wherein
a heat exchanger portion of the first of the at least two series-connected evaporators is connected to a media conduit, and where the media conduit is guided between the at least two series-connected evaporators such that a PACM-rich stream of matter is guided as a high boiler component in the bottoms of the at least two series-connected evaporators.

12. The plant according to claim 9, wherein a (substance) conduit for a solvent-rich stream of matter leads from a (tops) outlet of the first of the at least two series-connected evaporators to an inlet of the heat exchanger portion of the second of the at least two series-connected evaporators, and where a conduit leads from a bottoms outlet to
a (collection) vessel and/or
a connecting unit/node to/into the (return) conduit for the at least one solvent.

13. A process for catalytic hydrogenation of methylenedianiline (MDA; reactant1) with a hydrogen donor (reactant2), the process comprising:
effecting the catalytic hydrogenation by an industrial plant,
wherein
the industrial plant is designed according to claim 1.

14. The process according to claim 13, wherein a temperature of the reactant stream at the inlet of the main reactor is 80 to 135° C.

15. The process according to claim 13, wherein a pressure in the at least one main reactor is 60 to 120 bar.

16. The process according to claim 13, comprising:
implementing the process continuously and catalytically for production of methylenebis(cyclohexylamine).

17. The process according to claim 13, wherein
a temperature at an inlet of the at least one main reactor corresponds to a temperature at an inlet of the postreactor in a range or difference of +/−10° C., and/or
a pressure at the inlet of the at least one main reactor corresponds to a pressure at the inlet of the postreactor in a range or difference of +/−5 bar.

18. The process according to claim 13, wherein the MDA (reactant1) comprises a mixture of the following monomers:
4,4′ MDA,
2,4′ MDA, and
2,2′ MDA,
wherein a proportion of 4,4′ MDA is in a range from 75 to 98 mol %.

19. The process according to claim 13, wherein the first separation stage of the separation unit to the (first) separation tank comprises at least one further (second) separation tank disposed downstream of the first separation tank, where a bottoms outlet of the first separation tank is connected to an inlet of the second separation tank, where the first separation tank is operated at a first temperature and a first pressure, and the second separation tank is operated at a second temperature lower than the first temperature and a lower pressure compared to the first pressure.
